(19) United States
(12) Patent Application Publication
(10) Pub. No.: US 2010/0057100 A1
(43) Pub. Date: Mar. 4, 2010

(54) MULTIPLE BLADED SURGICAL KNIFE AND METHOD OF USE

(76) Inventor: Eli Zeevi, San Francisco, CA (US)

Correspondence Address: BUCHANAN, INGERSOLL & ROONEY PC, POST OFFICE BOX 1404, ALEXANDRIA, VA, 22313-1404, US

(21) Appl. No.: 12/202,989

(22) Filed: Sep. 2, 2008

(51) Int. Cl. A61B 17/322; A61B 17/32

(52) U.S. Cl. 606/133; 606/167

(57) ABSTRACT

A surgical knife having a handle having a distal end and a proximal end, the distal end including a base; a pair of movable blades removably mounted on the base of the handle; at least one spacer, which spaces the pair of blades from each other; and a shifting mechanism for longitudinally shifting the blades and the at least one spacer with respect to each other to a plurality of blade positions.

### FIELD OF THE INVENTION

The present invention relates to surgical knives, and more particularly to multiple bladed surgical knives with adjustable blades used in hair transplantation procedures.

### DESCRIPTION OF RELATED ART

Many hair transplantation procedures have been developed which transfer hair and living hair follicles from a donor to a donee. For example, a circular punch is commonly used to form a small diameter hole in the scalp of a donee.

Thereafter, a skin graft containing hair and follicles which has been removed from a donor is transplanted to the hole left by the punch. The size of the hole left by the punch often controls the number of hairs which may be transplanted in a single graft; usually, only a small number of hairs, such as 2 to 6 hairs are transplanted in a single graft. Sometimes, even a single hair is transplanted. Thus, a typical hair transplantation procedure requires a large number of grafts, which must be obtained from a donor. In some instances, hair is removed from and transplanted to the same individual.

Initially, grafts were obtained by carefully cutting sections of hair-laden scalp using a scalpel, which was a tedious and difficult procedure. In order to simplify the removal of grafts, knives with multiple parallel blades were developed such as the surgical knife as shown in U.S. Pat. No. 6,887,250 by Dority, et al. These known knives permit removal of long strips of hair-laden skin from a donor using a multiple bladed knives having three or more parallel blades enabling removal of more than one donor strip of skin in a single operation. When used to obtain skin strips from a donor's scalp, the knife is positioned against the donor's scalp, inserted to the desired depth and drawn along a number of centimeters, producing the above described strips of hair-laden skin. These strips are then cut into the numerous individual grafts typically required for hair transplantation to a donee.

It can be appreciated that hair customarily grows at an angle with respect to the surface of the scalp. Likewise, the hair follicles located below the skin are also at an angle with respect to the surface of the scalp. When removing strips of hair-laden skin, the knife edges of a multiple bladed knife when positioned perpendicular to the donor's scalp often inadvertently destroy hair follicles located below the scalp because they are located within the blades' path below the skin even though the hair itself may not be within the blades' path above the skin. In some instances, only 20% of the hair follicles on a donor strip remain intact for successful transplantations.

Alternatively, surgeons removing strips of skin with multiple bladed knives often orient the blades parallel to the direction of hair and follicle growth to minimize the chance that follicles may be destroyed. By angling the knife blades parallel with the direction of hair growth, less hair follicles are destroyed when the knife blades are drawn through the scalp tissue. This procedure permits a surgeon to cut a larger number of individual skin grafts from fewer donor strips.

However, angling the knife in such a manner generally produces undesirable results because angulation naturally forces some blades to penetrate the scalp further than others. More specifically, if the knife is angled such that a lower surface of the handle is nearest the scalp, the blades closest to the lower surface will penetrate the scalp further than the blades farthest from the lower surface. Such angled blades could penetrate the scalp too deeply, risking damage to adjacent follicles, and may unnecessarily injure the donor by cutting into the donor's skull. Likewise, too shallow blade penetrations are insufficient to properly remove the hairs and follicles.

Typically, the direction of hair growth with respect to the scalp changes in different areas of the scalp of a single donor. Because of these changes, the surgeon must continually re-evaluate the orientation of the hairs growing in the scalp. As the direction of the hairs change, the surgeon must readjust the orientation of the knife relative to the hairs such that the blades remain generally parallel to the hairs at all times during translation. This procedure must be done to prevent damage to a large number of follicles located within and outside the donor strip. When using the knives, the surgeon must continually approximate the depth that each of the blades should penetrate the scalp to achieve adequate depth penetration so that the entire follicle is removed while preventing contact with the skull.

Alternatively, it would be desirable to have a multiple bladed surgical knife, which has a means of controlling the depth of blade penetration, and more preferably only scores the scalp to a depth of approximately 0 to 1.5 mm, and also includes a shifting mechanism which permits longitudinal shifting of the forward ends of the blades with respect to each other and with respect to a base. In addition, it would also be desirable to provide a bladed surgical knife having blades with forward ends which define a line, and wherein the line defines an adjustable angle with respect to a longitudinal axis of the blades.

Still other objects and advantages of the present invention will become readily apparent to those skilled in the art from the following detailed description, wherein only the preferred embodiments of the present invention have been illustrated. As will be realized, the invention is capable of other and different embodiments, and its several details are capable of modification in various obvious aspects, all without departing from the invention. Accordingly, the drawings and description are to be regarded as illustrative in nature, and not restrictive.

### SUMMARY OF THE INVENTION

In accordance with an exemplary embodiment, a surgical knife comprises: a handle having a distal end and a proximal end, the distal end including a base; a pair of movable blades removably mounted on the base of the handle; at least one spacer, which spaces the pair of blades from each other; a securing member removably securing the blades and the at least one spacer to the handle; and a shifting mechanism for longitudinally shifting the blades and the at least one spacer with respect to each other to a plurality of blade positions.

In accordance with another exemplary embodiment, a method of scoring hair-laden tissue, comprises the steps of: providing a multiple bladed surgical knife having a pair of blades and at least one spacing element positioned between the pair of blades; determining a hair angle between a direction of hair growth and a line perpendicular to a scalp; shifting the blades to a plurality of positions whereby a line formed by a forward end of each of the blades is orientated at a blade angle with respect to a line perpendicular to a longitudinal axis of the knife, the blade angle being substantially equal to the hair angle; inserting the blades into the scalp at a direction parallel to the direction of hair growth; translating the blades through the scalp and scoring the scalp; and removing a hair-laden tissue strip from the scalp.

### BRIEF DESCRIPTION OF THE DRAWINGS

FIG. 1 is a perspective view of an exemplary embodiment of multiple bladed surgical knife in accordance with an embodiment.

FIG. 2 is an exploded perspective view of the multiple bladed surgical knife of FIG. 1.

FIG. 3 is a cross-sectional side view of the multiple bladed surgical knife of FIG. 1 in use.

FIG. 4 is a plan view of a knife blade in accordance with an embodiment.

FIG. 5 is a plan view of a spacer or spacing element in accordance with an embodiment.

FIG. 6 is a plan view of an extender in accordance with an embodiment.

### DETAILED DESCRIPTION OF THE PREFERRED EMBODIMENTS

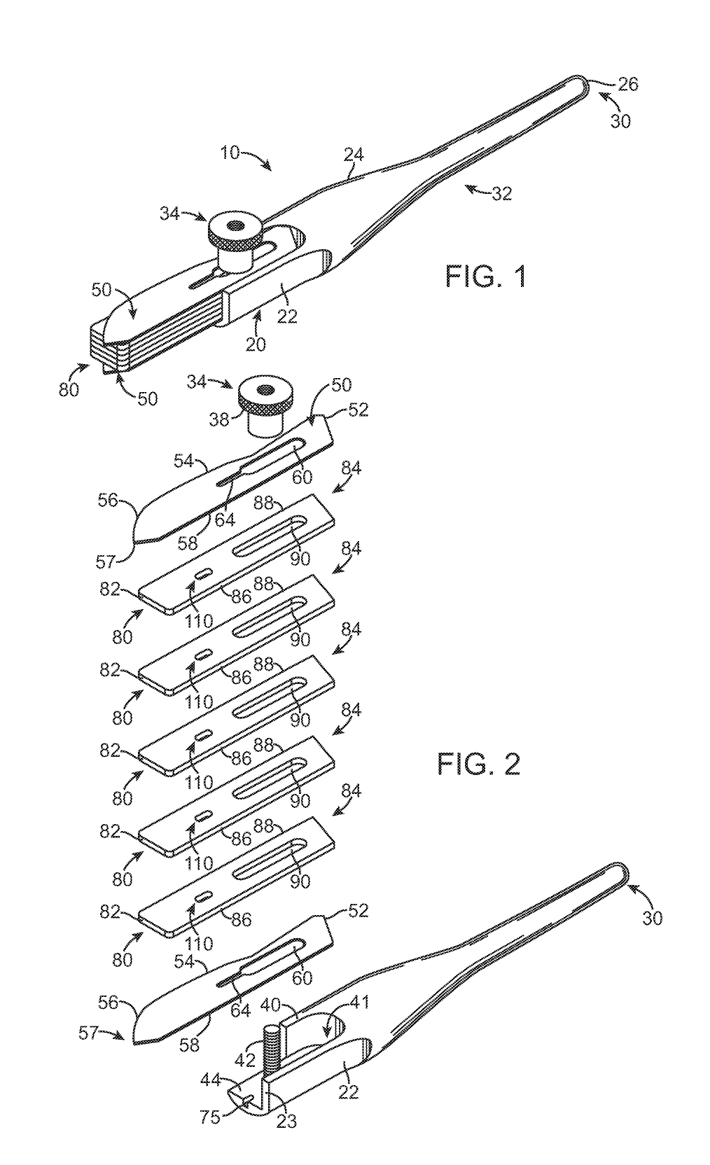

FIGS. 1 and 2 depict an exemplary embodiment of the present invention which includes a multiple bladed surgical knife 10 for scoring of a hair-laden skin strip from a donor's scalp. This donor strip is subsequently removed from a donor's scalp and cut into individual grafts for transplantation to a donee's scalp. An exemplary embodiment of the multiple bladed surgical knife 10 is shown in the perspective view of FIG. 1 and in the exploded view of FIG. 2. As shown in FIGS. 1 and 2, the multiple bladed surgical knife 10 includes a handle 32, a pair of surgical blades 50, and at least one spacer or spacing element 80 positioned between the pair of blades 50. In accordance with an exemplary embodiment, the spacer or spacing element 80 limits the depth of penetration of the pair of surgical blades 50 into the scalp, and more preferably the pair of surgical blades 50 score or penetrate the scalp to a depth of between 0 and 1.5 mm. In accordance with another exemplary embodiment, the pair of blades 50 can be set or spaced about to produce a strip of hair-laden tissue from the scalp, which measures between about 1 mm to 20 mm.

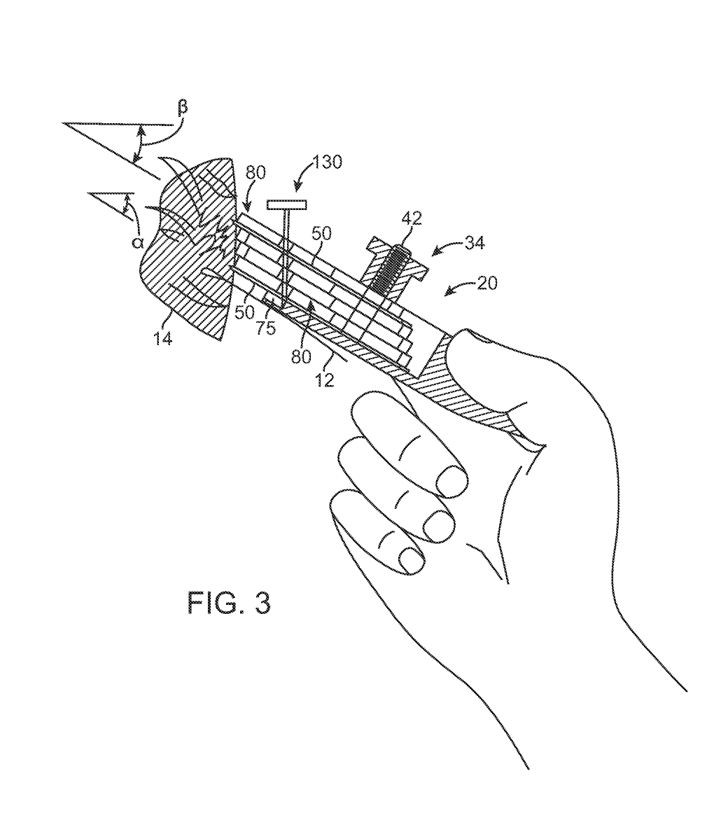

In accordance with an exemplary embodiment, each of the surgical blades 50 has a sharpened forward end 57 and are spaced apart from each other by at least one spacer or spacing element 80. A securing member such as a nut 34, for example, secures the blades 50 to the handle 32. As will be described below with reference to FIG. 3, the blades 50 may advantageously shift longitudinally with respect to each other at a surgeon's discretion such that the forward ends 57 of the blades 50 may change positions in a unique manner. This adjustability permits scoring of donor strips of consistent depth while minimizing the number of hair follicles that are destroyed during donor strip removal.

As shown in FIG. 1, the multiple bladed surgical knife 10 comprises a handle 32 having a proximal end 30 and distal end 20. It can be appreciated that since the knife 10 will be used in surgical environments, the handle 32 is preferably made of stainless steel, which is non-corrosive, easy to clean, and provides proper knife balance. Although stainless steel is the preferred material of the handle 32, other non-corrosive materials are contemplated such as various plastics and resins. The proximal end 30 of the handle 32 includes a smooth and tapered end surface 26, which prevents the handle 32 from unintentionally puncturing items during surgery. The handle 32 also includes a tapered portion 24 between the proximal end 30 and the distal end 20. The tapered portion 24 preferably gives the knife 10 a comfortable ergonomic feel during use in surgery.

As shown in FIGS. 1 and 2, the distal end 20 of the knife handle 32 includes a recess 41 for receiving the pair of blades 50 and at least one spacer or spacing element 80. The blades 50 are preferably a standard stainless steel type blade commonly used in the surgical industry, and are relatively thin, as compared to the at least one spacer or spacing element 80. As shown in FIGS. 1 and 2, when the blades 50 are placed in the recess 41, with the at least one spacer or spacing element 80, and more preferably a plurality of spacers or spacing elements 80 located between the blades 50. In accordance with an exemplary embodiment, the spacers or spacing elements 80 thickness varies from 0.25 to 5.0 millimeters, preferably from 0.75 to 3.5 millimeters, but may vary outside this range depending upon a surgeon's needs. Thus, the blades 50 are spaced apart by the thickness of the individual spacers or spacing elements 80.

In accordance with an exemplary embodiment, and as shown in FIG. 2, each of the blades 50 contain an elongated rear opening 60 extending in the longitudinal direction of the blades 50. The spacers or spacing elements 80 include an elongated bolt opening 90 extending in the longitudinal direction of the spacers or spacing elements 80. These two openings 60 and 90 in each of the blades 50 and spacers or spacing elements 80 are adapted to receive a bolt 42 which is attached to a base 44 at the distal end 20 of the handle 32. The base 44 forms the bottom of the recess 41. The base 44 is substantially flat and preferably perpendicular to the inner surface of a forward wall 23 of the recess 41.

As shown in FIG. 2, the bolt 42 preferably extends perpendicularly to the base 44 and receives a nut 34. The nut 34 has female threads adapted to receive the male threads of the bolt 42. The nut 34 also has an enlarged knurled exterior portion 38 with a series of small grooves and ridges to aid the surgeon in gripping and twisting the nut 34. Thus, in assembling the knife 10, a first bottom blade 50 is oriented such that the opening 60 receives the bolt 42. The first blade 50 is then lowered into the recess 41 and placed on the base 44 such that a top edge 58 of the blade 50 is immediately adjacent an inside surface of the forward wall 23. The blades 50 each have a cutting edge 56 which curves from a bottom edge 54, located opposite the top edge 58, to the blade's forward end 57. It can be appreciated that, the blades 50 are oriented in the recess 41 such that the cutting edge 56 faces generally away from the inside surface of the forward wall 23.

Next, at least one spacer or spacing element 80 is oriented such that the bolt opening 90 of the spacer or spacing element 80 receives the bolt 42. The at least one spacer or spacing element 90 is then lowered into the recess 41. The recess 41 is preferably shaped and the bolt 42 is preferably located such that the spacers or spacing elements 80 and blades 50 fit squarely within the area between the forward wall 23 and a rearward wall 40. Thus, once the spacers or spacing elements 80 and a second blade 50 are placed within the recess 41, there is minimal room for rotation of the spacers and blades about the bolt 42. This configuration minimizes movement of the blades 50 during surgery.

The forward wall 23 abuts against the top edge 58 of the blades 50 and a corresponding top edge 86 of the spacer or spacing element 80. This abutting relationship prevents the blades 50 and spacers or spacing elements 80 from rotating about the bolt 42, especially when the blades 50 and spacers or spacing elements 80 are in a shifted position. Thus, when the blades 50 score the scalp 14, the forward wall 23 supports the blades 50.

After the first blade 50 and the first of the at least one spacer or spacing element 80 is placed in the recess 41 in the manner described above, additional spacers or spacing elements 80 can be similarly located, a number of which depends on a surgeon's preference and a second blade 50 is placed in the recess 41. As shown in FIG. 2, one embodiment of the present invention has a pair of blades 50, and four spacers or spacing elements 80.

Once the chosen number of blades 50 and spacers or spacing elements 80 have been located within the recess 41, the blades 50 are then secured in place by a securing member, which in one embodiment of the present invention is the nut 34 and bolt 42 illustrated in FIGS. 1 and 2. Although a nut 34 and bolt 42 are the preferred means of securing the blades 50 and spacers or spacing elements 80 to the handle 32, a number of different means for securing the blades to the handle 32 are contemplated by the present invention. For example, the blades 50 may be held in place by any securing member or means for securing, such as self tapping screws, wood screws, machine screws, split screws, any nut and bolt configuration, pressure clasps, snaps, buckles, pins, latches, straps, or snaps. The nut 34 and bolt 42 are preferred because a surgeon may easily and dependably remove, loosen or tighten the nut and blades 50 on demand.

As shown in FIGS. 1 and 2, the pair of blades 50 may be positioned such that the blade forward ends 57 are all located along a common line. In one instance as shown in FIG. 1, the blades 50 form a line perpendicular to the longitudinal axis of the handle 32; this is called the zero degree position. Because the blades 50 are preferably identical, when the blades 50 are located in the zero degree position, the back edges 58 of the blades 50 are all located at the same position in the longitudinal direction of the handle 32, as are the blade forward ends 57, i.e., in one stationary position such that the blades 50 do not move longitudinally with respect to one another.

As shown in FIG. 3, the pair of blades 50 (and the spacers or spacing elements 80) can be longitudinally shifted with respect to each other to a plurality of positions whereby the forward ends (or tips) 57 of the blades 50 (and the forward ends 82 of the spacers or spacing elements 80) form a plurality of different angled lines. For example, one line is formed in the zero degree position illustrated in FIG. 1 and a second line is formed at the angled position of FIG. 3. This shifting is accomplished by a shifting mechanism which is further described hereinafter.

As illustrated in FIG. 3, the blade ends 57 and spacers or spacing elements 80 may be positioned to form a line at a blade angle β with respect to a line y perpendicular to the planes of the blades 50 and the spacers or spacing elements 80. The blade angle β can also be measured between a line z perpendicular to the line formed by the blade ends 57 (forward end 83 of the spacers or spacing elements 80), and the plane of the blades 50. The range of blade angles β is from 0 to 75 degrees, preferably 0 to 45 degrees. The largest blade angle β permitted depends upon the size of the bolt 42, the bolt opening 90, and the rear opening 60, which may be any variety of dimensions and still be within the present invention.

In accordance with an exemplary embodiment, the base 44 at the forward edge of the recess 41 includes an pivoting point or dimple 75, which is configured to receive the tip of the adjustment pin 130, and simplifies the angle adjustment. Further, the blades 50 have an additional elongated forward opening 64, and the spacers or spacing elements 80 have an additional pin opening 110. Both openings 64, 110 are adapted to receive a pin 130 as shown in FIG. 3.

When the blades 50 are placed into the knife recess 41 and secured to the knife 10 by the nut 34 in the zero degree position, the pin 130 may then be vertically placed through the forward openings 64 and the pin openings 110 such that a tip of the pin 130 rests in the pivoting point or dimple 75 as shown in FIG. 3. Thereafter, the pin 130 is angled with respect to the longitudinal axis of the knife 10 such that the blades 50 and spacers 80 longitudinally shift distally with respect to each other and with respect to the base 44 as the pin 130 is rotated in the longitudinal direction of the knife 10.

As shown in FIG. 3, the pin 130 and the openings 60, 64, 90 and 110 cause the blade ends or tips 57 and the forward ends 82 of the spacers or spacing elements 80 to longitudinally shift to a plurality of positions in accordance with an exemplary embodiment. The forward opening 64, the pin opening 110, the pin diameter, pin length, and the pivoting point or dimple 75 are all sized such that above described range of blade angles β may be achieved by rotating the pin 130 about the pin tip in the pivoting point or dimple 75. These aforementioned sizes may be in any variety of combinations and are easily determined by experimentation or geometrical design, as is commonly known in the art. The pin 130 which is employed for shifting the blades 50 and the spacers or spacing elements 80 may be a conventional hypodermic needle. Once the blades 50 and the spacers or spacing elements 80 are moved into an angled position as illustrated in FIG. 3, the surgeon need only retighten the nut 34, securing the blades 50 and the spacers or spacing elements 80 in the new angled position.

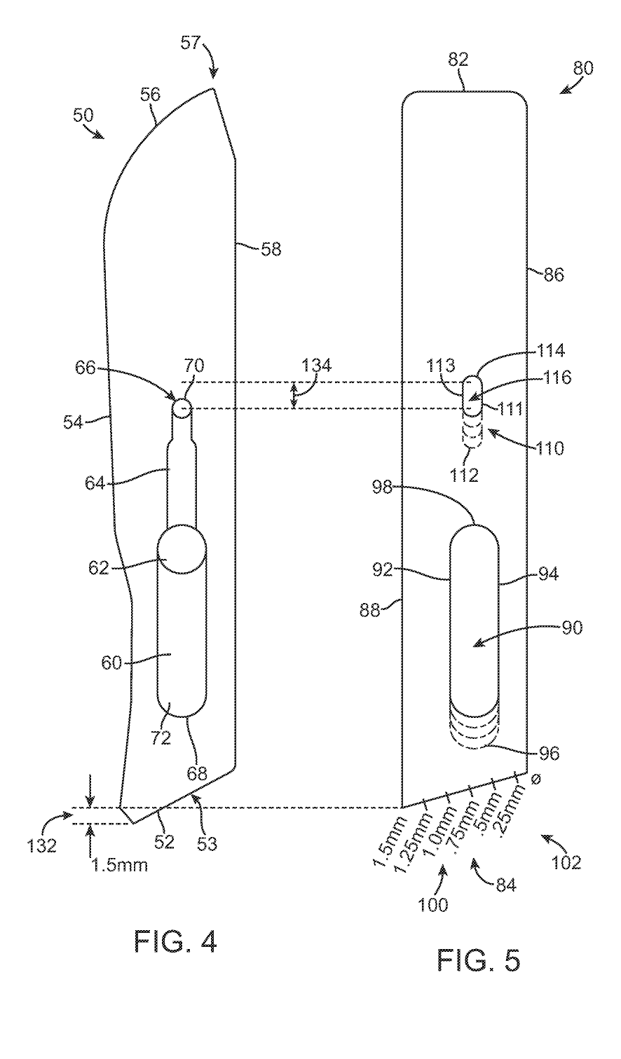

FIG. 4 is a plan view of a knife blade 50 in accordance with an embodiment. As shown in FIG. 4, the knife blade 50 includes a back end 52, a bottom edge 54 located opposite the top or back edge 58, a cutting edge 56 and a forward end or tip 57. The blades 50 each have a cutting edge 56 which curves from a bottom edge 54, located opposite the top edge 58, to the blade's forward end 57. The blades 50 are oriented in the recess 41 such that the cutting edge 56 faces generally away from the inside surface of the forward wall. The back end 52 has an angular surface which in combination with an angular surface 100 on the back end 84 (FIG. 5) of the spacer or spacing element 80 provides a means for determining the depth of penetration of the forward end or tip 57 into the scalp 12.

The knife blade 50 also includes an elongated rear opening 60 and an additional elongated forward opening 64, which are in communication with one another. The elongated rear opening 60 includes a forward opening 62, which is generally circular in shape and configured to receive the bolt 42 from the handle of the knife 10. A rear opening 72 having a rear wall 68 limits the movement of the bolt 42 upon adjustment of the blades 50 and spacers or spacing elements 80. The forward opening 62 also includes a forward opening 66, which is generally circular in shape and is configured to receive a portion of the pin 130 during adjustment of the blades 50 and spacers or spacing elements 80. The forward opening 66 has a forward wall 70, which defines an upper edge of the forward opening 62.

FIG. 5 is a plan view of a spacer or spacing element 80 in accordance with an embodiment. As shown in FIG. 5, the spacer or spacing element 80 includes a back end 84, a bottom edge 88 located opposite the top or back edge 86, and a forward end or tip 82. The spacer or spacing element 80 is oriented in the recess 41 between the pair of blades 50 such that the bottom edge 88 faces generally away from the inside surface of the forward wall 23. The forward end or tip 82 can be essentially square, square with round outer edges or can have a radius thereto without departing from the present invention. In accordance with an exemplary embodiment, the back end 84 has an angular surface 100 which in combination with an angular surface 53 on the back end of the blade 50 provides a means for determining the depth of penetration of the forward end or tip 57 of the blade 50 into the scalp.

The spacer or spacing element 80 also includes an elongated rear opening 90 and an additional elongated forward opening 110. The elongated rear opening 90 is configured to receive the bolt 42 from the handle of the knife 10. The rear opening 90 has a pair of side walls 92, 94, a lower edge 96 and an upper edge 98. The lower edge 96 and the upper edge 98 limits the movement of the bolt 42 upon adjustment of the blades 50 and spacers or spacing elements 80. The elongated forward opening 110 includes an elongated opening 116 having a lower edge 112, an upper edge 114 and a pair of side walls 111, 113. The elongated forward opening 110 is configured to receive a portion of the pin 130 during adjustment of the blades 50 and spacers or spacing elements 80.

As shown in FIGS. 4 and 5, the rear openings 60 of the blades 50 and the bolt openings 90 of the spacers or spacing elements 80 are sized to receive the bolt 42, but are also sized such that the spacers or spacing elements 80 and the blades 50 can be longitudinally shift along the bolt. Thus, each of the blades 50 and the spacers 80 may be shifted to any number of longitudinal positions between the two extreme positions permitted by the rear opening 60 and the bolt opening 90. It can be appreciated that depending on the thickness of the spacer or spacing element 80, the side of the elongated rear opening 90 and the elongated forward opening 110 will vary, such that as the thickness of the spacer or spacing element 80 increases, the size of the elongated forward and rear openings 90, 110 also increase in size.

In accordance with an exemplary embodiment, the angular surface 100 has a series of markings 102, which depict the difference between the forward ends 57 of the blades 50 and the spacer or spacing elements 80. For example, in accordance with an exemplary embodiment the markings 102 can be from 0.0 mm to 1.5 mm. It can be appreciated that the relative difference between the forward ends 57 and the at least one spacer or spacing element 80 is not limited to 1.5 mm and other relative differences can exist, for example, 0 to 5.0 mm or greater.

Although the embodiments of FIGS. 4 and 5 depicts that the pin opening 110 is an elongated circular opening, it need not be shaped precisely so. For example, the bolt opening 90 and the pin opening 110 may be one continuous opening, similar to the openings 60 and 64 in the blades 50. Likewise, the openings 60, 64 in the blades 50 may be shaped and size similar to the openings 90, 110 in the spacers or spacing elements 80. The shape and size of the openings 60, 64, 90, and 110 may vary, assuming such permit the blades 50 and the at least one spacer or spacing elements 80 to shift, and still fall within the bounds of the present invention.

It can be appreciated that one of the advantages of using an exemplary embodiment of the present invention is that hair and hair follicles grow at an angle a with respect to a line perpendicular to the scalp. Because of this angling of the hair, a surgeon must carefully position the longitudinal axis of the multiple bladed knife 10 at an angle substantially equal to the angle a of the hair such that the knife and hair are generally parallel. By doing this, the percentage of hair follicles destroyed under the skin is minimized when a surgeon cuts the donor strips from the scalp; if this is not done, the blades 50 may destroy a majority of the hair follicles, which then requires that more donor strips be removed to obtain the necessary number of grafts for the resulting hair transplantation operation.

However, once a knife 10 having the blade forward ends 57 in the zero degree position (where the blade angle β equals zero degrees), is angled parallel with the hair growth and inserted into the scalp, some blades 50 will penetrate the scalp further than others. Thus, it is advantageous to insert the pin 130 into the openings 64 and 110, and then longitudinally shift the blade tips 57 such that the line formed by the blade tips forms a blade angle β substantially equal to the hair angle α.

By angling the blades at the blade angle β and angling the knife 10 at an angle generally parallel with the hairs, donor strips of uniform depth may be removed without damaging the follicles or injuring the donor. Furthermore, when the surgeon selects a section of hair for donor strip removal which has an average hair angle α. different than a previous area, the surgeon may conveniently readjust the blade angle β in the manner described above. Readjustment may be completed between separate donor strip removal operations. Hence, a surgeon may remove donor strips using only a single knife 10, which can accommodate a multitude of hair angles α, while also minimizing hair follicle damage and keeping the donor strips a consistent depth.

In accordance with an exemplary embodiment, the spacers or spacing elements 80 are configured such that when the blades 50 are inserted into the scalp 14, the forward ends 82 of the spacers or spacing elements 80 will abut against the scalp 14, preventing further penetration of the blades 50. In accordance with an exemplary embodiment, the spacers or spacing elements 80 have a length l between the opening 110 for receiving the pin 130 and the forward end 82 of the spacer or spacing element 80. The length l is sized to limit the penetration depth d of the forward end of the blade 50 from 0 to 1.5 millimeters. The depth limiting function of the spacers 80 provides a benefit which permits the surgeon to concentrate on the knife and blade orientation, without being entirely concerned with the blade penetration depth d.

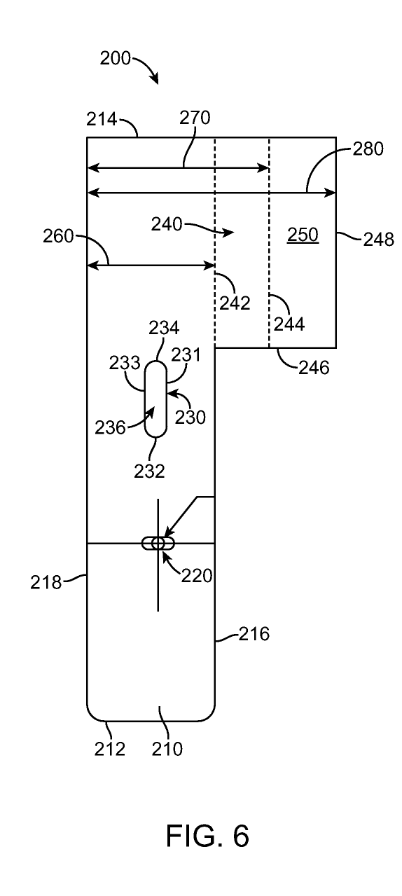

FIG. 6 is a plan view of an extender 200 in accordance with another embodiment. The extender 200 is configured to fits on an opposite side of the forward wall 23 of the handle 20 to hold the blades 50 and the at least one spacer 80 in place during use when a distance from one blade 50 to the other blade 50 is greater than the width or depth of the recess 41. As shown in FIG. 6, the extender 200 includes screw side portion 210, a top portion 240, which is perpendicular to the side portion 210, and a forward wall portion 250, which is perpendicular to the side portion 210. The top portion 240 fits over the upper edges 58, 86 of the blades 50 and the spacer or spacing elements 80. The forward wall portion 250 fits over an opposite outer surface of the blades 50 and the at least one spacer or spacing element 80. The forward wall portion 250 is configured to match a forward wall portion 22 (FIG. 2) of the knife 10. The top portion 240 and forward wall portion 250 are shown in FIG. 6 in the same plane as the side portion 210, however, in reality the top portion 240 is perpendicular (i.e., 90 degrees to the side portion 210) having a fold line 242, and the forward wall portion 250 is perpendicular (i.e., 90 degrees to the top portion 240) having a fold line 244.

The side portion 210 has a top or back edge 216, a bottom edge 218 located opposite the top or back edge 216, and a forward end or tip 214, and a back end 212. The side portion 210 is oriented in the recess 41 such that the bottom edge 216 faces generally away from the inside surface of the forward wall. The extender 200 also includes an elongated rear opening 220 and an additional elongated forward opening 230. The elongated rear opening 220 is configured to receive the bolt 42 from the handle of the knife 10. The elongated forward opening 230 includes an elongated opening 236 having a lower edge 232, an upper edge 234 and a pair of side walls 231, 233. The elongated forward opening 230 is configured to receive a portion of the pin 130 during adjustment of the blades 50 and spacers or spacing elements 80.

The top portion 240 and the forward wall portion 250 extend from the forward end or tip 214 to a lower edge 246. In accordance with an exemplary embodiment, a distance 260 from the bottom edge portion 218 to the fold line 242 is approximately 12 mm. In addition, a distance 270 from the bottom edge portion 218 to the second fold line 244 is 16 mm and a distance 280 from the bottom edge portion 218 to an outer edge 248 of the forward wall portion 250 is 20 mm. It can be appreciated that based on the size of the blades 50 and the at least one spacer or spacing element 80, the distance from the bottom edge portion 218 to the fold line 242, the second fold line 244 and the outer edge 248 can be varied to accommodate a knife 10 having blades 50 and spacers or spacing elements 80 of different sizes. In accordance with an exemplary embodiment, the pair of blades 50 can be set or spaced about to produce a strip of hair-laden tissue from the scalp, which measures between about 1 mm to 20 mm. It can be appreciated that for strip of hair-laden tissues having a width of 10 mm or greater, the extender 200 can be used to secured the blades 50 and spacers or spacing elements 80 within the handle 20 of the knife, such that during scoring of the scalp 12 the blades 50 and the spacers or spacing elements 80 remain in a fixed position.

It can be appreciated that other mechanisms for shifting the blades can be used. For example, the shifting of the blades may be performed by gravity, specially adapted springs, variously shaped magnets, levers, pistons, rods, or other similar apparatus commonly known in the art. Although the previous described embodiments of the present invention use standard blades 50 which are known in the surgical industry, other blades may be used which are not of standard sizes. By using blades of different sizes having differently sized openings 60 and 64, different maximum blade angles β may be achieved without angling the bolt 42.

The above described embodiments of the present invention are used to score donor strips from a donor for transplantation to a donee, who are often the same individual. In doing so, a surgeon using the above describe multiple bladed surgical knife would: (1) determine the hair angle a the hair 12 makes with respect to a line perpendicular to the scalp 14, (2) shift the blades 50 to a plurality of positions such that the blade angle β is substantially equal to the hair angle a, (3) insert the blades into the scalp parallel with the hair angle α, (4) translate the blades through the scalp to score the area, and finally (5) remove the hair-laden donor strip from the scalp.

To realize another important benefit of the invention, using the spacer or spacing elements 80 prevent the blades 50 from penetrating the tissue beyond the predetermined blade penetration depth, as described above, a surgeon would also: (1) determine the blade penetration depth d, (2) choose a plurality of spacers or spacing elements 80 for installation into the knife 10, and (3) shift the forward ends 82 of the spacer or spacing elements 80 to a plurality of positions located at a distance from each the forward ends 57 of the blades 50 substantially equal to the determined blade penetration depth.

Each of the forgoing observations are results of the present invention. The above description of the preferred embodiments of the present invention must be considered as illustrative only of the principle of the invention and not limitative. Indeed, it may be easily understood that numerous modifications could be made by those skilled in the art without departing from the spirit of the invention as defined in the claims below.

What is claimed is:

1. A surgical knife comprising:
a handle having a distal end and a proximal end, the distal end including a base;
a pair of movable blades removably mounted on the base of the handle;
at least one spacer, which spaces the pair of blades from each other;
a securing member removably securing the blades and the at least one spacer to the handle; and
a shifting mechanism for longitudinally shifting the blades and the at least one spacer with respect to each other to a plurality of blade positions.

2. The surgical knife according to claim 1, wherein each of the blades having a longitudinal axis, a sharpened forward end and an angular back end, and the at least one spacer having an upper end and a lower edge having sequential angular markings on the lower edge, which in combination with the angular back end provides a relative relationship between the front end of the blades and the upper end of the at least one spacer

3. The surgical knife according to claim 2, wherein the each of the front edges of the at least spacers are located at a predetermined distance from each of the forward ends of the blades, and whereby the front edges of the at least one spacer prevents the blades from scoring tissue beyond 1.5 mm.

4. The surgical knife according to claim 2, wherein the forward ends of the blades forming a first line at a first blade position and a second line at a second blade position, and the first line forms a first angle with respect to the longitudinal axis of the blades, and the second line forms a second angle with respect to the longitudinal axis of the blades.

5. The surgical knife according to claim 1, wherein each of the spacers further includes a front end and a longitudinal axis, wherein the shifting mechanism longitudinally shifts the spacers with respect to each other to a plurality of spacer positions, the front edges of the spacers forming a first spacer line at a first spacer position and a second spacer line at a second spacer position.

6. The surgical knife according to claim 5, wherein the first spacer line forms an initial angle with respect to the longitudinal axis of the spacers, and the second spacer line forms an angle greater than the initial angle with respect to the longitudinal axis of the spacers.

7. The surgical knife according to claim 3, wherein the longitudinal axis of the spacers is substantially parallel with the longitudinal axis of the blades.

8. The surgical knife according to claim 1, wherein each of the spacers and the blades further includes an elongated opening extending in a longitudinal direction of the blades and the spacers, and wherein the elongated openings in the blades and the spacers receives the securing member.

9. The surgical knife according to claim 8, wherein the blades and the spacers shift when shifted by the shifting mechanism longitudinally with respect to the securing member.

10. The surgical knife according to claim 1, wherein the securing member includes a screw attached to the base of the handle and a nut threaded on the screw.

11. The surgical knife according to claim 1, wherein the base of the handle includes a recess for receiving a portion of the blades and the at least one spacer within the distal end of the handle.

12. The surgical knife according to claim 1, wherein the shifting mechanism includes a pin extending through openings in the blades and the spacers for adjusting relative positions of the blades.

13. The surgical knife according to claim 12, wherein the base of the handle includes a pivoting point for receiving a tip of the pin and the relative positions of the blades are adjusted by pivoting the pin about the pin tip received in the pivoting point.

14. The surgical knife according to claim 1, further including a forward wall abutting a portion of each of the blades and the at least one spacer, the forward wall supporting the blades and the at least one spacer in a shifted position.

15. The surgical knife according to claim 1, further including a forward wall abutting a portion of each of the blades and the at least one spacer, the forward wall preventing rotation of the blades and the at least one spacer.

16. The surgical knife according to claim 1, further comprising an extender, which fits on an opposite side of the forward wall to hold the blades and the at least one spacer in place during use.

17. The surgical knife according to claim 16, wherein the extender comprises a screw side portion, a top portion, which is perpendicular to the side portion, and a forward wall portion, which is perpendicular to the side portion.

18. The surgical knife according to claim 17, wherein:
the top portion fits over the upper edges of the blades and the spacer or spacing elements;
the forward wall portion fits over an opposite outer surface of the blades and the at least one spacer or spacing element; and
the forward wall portion is configured to match a forward wall portion of the knife.

19. A method of scoring hair-laden tissue, comprising the steps of:
providing a multiple bladed surgical knife having a pair of blades and at least one spacing element positioned between the pair of blades;
determining a hair angle between a direction of hair growth and a line perpendicular to a scalp;
shifting the blades to a plurality of positions whereby a line formed by a forward end of each of the blades is orientated at a blade angle with respect to a line perpendicular to a longitudinal axis of the knife, the blade angle being substantially equal to the hair angle;
inserting the blades into the scalp at a direction parallel to the direction of hair growth;
translating the blades through the scalp and scoring the scalp; and
removing a hair-laden tissue strip from the scalp.

20. The method according to claim 19, further comprising the steps of:
determining a blade scoring depth of between approximately 0.0 mm and 1.5 mm; and
shifting the at least one spacing element to a position whereby a distal end of the at least one spacing element is located a distance from each the forward ends of the blades substantially equal to the determined blade scoring depth, whereby the distal end of the at least one spacing element prevent the blades from penetrating the tissue beyond the determined blade scoring depth.
